(19) United States
(12) Patent Application Publication
(10) Pub. No.: US 2025/0361542 A1
(43) Pub. Date: Nov. 27, 2025

(54) Method and System for Producing an Object from a Vegetable Material

(71) Applicant: INM - Leibniz-Institut für Neue Materialien gemeinnützige GmbH, Saarbrücken (DE)

(72) Inventors: Wilfried Weber, St. Ingbert (DE); Maria Rosanne Schmachtenberg, Heidelberg (DE); Mateo Carl Laskowski, Freiburg (DE); Johannes Falkenstein, Neustadt a. d. W. (DE)

(21) Appl. No.: 18/874,632

(22) PCT Filed: Jun. 21, 2023
(86) PCT No.: PCT/EP2023/066759

§ 371 Date: Dec. 13, 2024

(30) Foreign Application Priority Data
Jun. 21, 2022 (DE) 10 2022 115 465.3

(51) Int. Cl. C12P 21/00; A47C 5/00; C12M 1/12; C12M 1/42; C12N 15/64

(52) U.S. Cl. CPC C12P 21/00; C12M 25/14; C12M 35/04; C12N 15/64; A47C 5/00

(57) ABSTRACT

A process (1) for producing an article (2) from a plant-based material (3) includes providing (4) genetically modified microorganisms (5) in which the genetic modification (12) results in overexpression of at least one adhesive protein (6); mixing (7), preferably blending, of the plant-based material (3) with the genetically modified microorganisms (5), which are preferably held in a culture medium (11), to obtain a mixture (8); expressing of the adhesive protein (6); and hardening (10) of the mixture (8).

The invention relates to a process for producing an article from a plant-based material.

Processes for producing articles from plant-based materials are known in particular from the furniture and construction industries. In known processes, piecemeal plant-based starting materials are commonly combined to form an article. In order to combine the starting materials together cohesively, formaldehyde-containing adhesives in particular are used. A disadvantage of this can be that formaldehyde, which is harmful to health, is given off by the finished articles and absorbed by the user.

Other known processes employ microorganisms, commonly yeasts or other fungi, in the manufacture of articles. Such microorganisms are usually wild types, i.e. microorganisms that have not been genetically modified. Genetic modification is here preferably understood as meaning the insertion and/or modification of at least one nucleic acid (sequence) and the permanent preservation of said at least one nucleic acid (sequence) in the genome of a microorganism. However, genetically modified microorganisms can also be produced in other ways known to those skilled in the art. The use of wild types in known processes is disadvantageous in that the wild types generally need to be mixed and incubated with the piecemeal plant-based starting materials for a long period, often several days. This is because, firstly, the wild types are often employed as pore-formers with the aim of lowering the density of an article being produced and, secondly, the wild types are utilized for the breakdown of components of the plant-based material. The efficiency and flexibility of wild types in the production of substances, especially for the processing and/or manufacture of articles, is likewise limited.

The invention further relates to an article produced from plant-based materials, which is preferably monolithic.

The invention further relates to a system for producing the article described and claimed herein.

The invention further relates to the use of genetically modified microorganisms for the microbial production of an adhesive protein.

It is therefore an object of the invention to improve the production of articles from plant-based material.

For the achievement of the stated object, the features of claim 1 are provided in accordance with the invention. In particular, to achieve the stated object in a process of the type described in the introduction it is thus proposed in accordance with the invention that the process comprises the following process steps: providing genetically modified microorganisms in which the genetic modification results in overexpression of at least one adhesive protein; mixing, preferably blending, of the plant-based material with the genetically modified microorganisms, preferably held in a culture medium, to obtain a mixture; expressing of the adhesive protein; hardening of the mixture. Providing genetically modified microorganisms makes it possible to produce at least one adhesive protein that results in adhesive bonding of components of the plant-based material. The expressed adhesive protein allows components of the mixture to be adhesively bonded. As a consequence of the genetic modification, during expression of the adhesive protein said protein is overexpressed. Through the process according to the invention, adhesive can be produced in the form of a microbial adhesive protein that is free of harmful substances, in particular formaldehyde. An article produced by the process according to the invention can thus be environmentally friendly and in particular recyclable. The article can likewise be incinerated or composted, which means there is little or no release of substances harmful to health.

The abovementioned process steps may be carried out consecutively, but they may also be carried out simultaneously or non-consecutively. For example, the overexpression of the adhesive protein may already be taking place before the plant-based material is mixed with the genetically modified microorganisms. It is however also possible that overexpression does not take place until after mixing.

The employed genetically modified microorganisms may be any archaea, bacteria and/or unicellular eukaryotes known to those skilled in the art. Mycelium-forming fungi can likewise be used. Genetically modified species of the genuses Escherichia, Bacillus, Caulobacter, Saccharomyces and/or Sporosarcinia in particular may be used. Species of these genuses in particular are easy to genetically modify and to culture. This makes it possible for a process to be particularly cost-effective and customized.

The use of the microbially produced adhesive protein allows the process according to the invention to be quick and cost-effective. Because the process is particularly well suited to the use of recyclable plant-based waste materials, for example wooden furniture, an article can have a particularly favorable ecological footprint.

An adhesive protein can in particular be a protein that is homologously or heterologously overexpressed in the genetically modified microorganisms. An adhesive protein may in particular be at least one of the following: MFP (mussel foot protein) and MFP fusion proteins and derivatives thereof having the characteristic feature of a high tyrosine content; surface proteins and/or microbial surface structures, particular preferably curli fibers, TasA and Bs1A of Bacillus subtilis and TasA/Bs1A homologs; synthetic proteins, particularly preferably ELPs (elastin-like polypeptides); fibrous proteins and also polysaccharide- and cellulose-binding proteins. Synthetic and/or hybrid proteins, particularly hybrids of the abovementioned proteins, can also be used as adhesive protein. A large number of adhesive proteins can be produced in this way. This advantageously makes it possible to select an adhesive protein for a particular genetically modified microorganism, in order for it to be produced and formed (folded) by said microorganism in adequate amounts. This makes it possible for the efficiency of the process according to the invention to be particularly high.

A genetic modification can in particular be a genetic modification as described in the introduction. The genetic modification allows an adhesive protein to be selectively overexpressed and produced. The genetic modification in particular makes it possible to tailor and/or overexpress the adhesive protein in line with technical requirements. This makes it possible for the process according to the invention to be used with particular versatility for the production of articles from a plant-based material.

In the process it may be the case that the genetically modified microorganisms are precultured before being provided. In this case it may be advantageous when the adhesive protein is produced prior to mixing, so that the adhesive bonding of the mixture in particular may be carried out particularly quickly. This makes it possible to prevent undesired metabolization by the genetically modified microorganisms of plant-based constituents of the plant-based material and a reduction in the quality of the article.

In particular, the genetically modified microorganisms can be cultured such that the microorganisms achieve a cell count per desired volume of mixture that produces a sufficiently large amount of adhesive protein for adhesive bonding. The genetically modified microorganisms can in this case be cultured in a culture medium.

It may be the case that the genetically modified microorganisms are held in the culture medium and added to and mixed with the plant-based material. This makes it possible to at least maintain the viability of the genetically modified microorganisms and/or the overexpression of the at least one adhesive protein. The process according to the invention accordingly makes it possible to produce articles from a plant-based material particularly efficiently.

It may also be the case that the genetically modified microorganisms and/or the adhesive proteins are concentrated before being provided. This can be carried out in particular by centrifugation and/or filtration and/or other methods known to those skilled in the art. Concentration allows a desired cell count of genetically modified microorganisms and/or adhesive proteins to be achieved, so that sufficient microorganisms and/or protein material are available for mixing and adhesive bonding.

In the process it may also be the case that the article is elaborated after hardening. Elaboration of the article is to be understood as meaning in particular that the article is sanded and/or drilled and/or sawn and/or milled and/or dowelled and/or screwed and/or glued and/or coated and/or painted. The article may also be connected to other materials after hardening, particularly when the article is not monolithic. Elaboration allows the article to be optimally customized to its intended use and/or to customer requirements.

The mentioned process steps and associated advantages allow the production of articles from plant-based material to be improved.

In an advantageous embodiment it may be the case that the genetic modification encompasses a gene sequence that codes for an adhesive protein. Such a gene sequence can accordingly be advantageously inserted into a genetically modified microorganism and, as a result, heterologous overexpressions in a particular genetically modified microorganism are also enabled. The gene sequence may also include regulatory elements, for example promoter elements, used for overexpression of the adhesive protein. The advantage of promoter elements is that they are able to respond to particular stimuli, as a result of which the overexpression of a protein is regulated. This makes it possible, through promoter elements and the administration of a stimulus, in particular as described and/or claimed hereinbelow, to control the overexpression of an adhesive protein and/or of a substance as described herein.

Promoter elements can be any promoter elements known to those skilled in the art. Particular preference is given to the use of promoter elements that respond in particular to electromagnetic radiation and/or sugar derivatives and/or primary metabolites and/or quorum-sensing substances and/or antibiotics and derivatives thereof and/or thermal radiation.

In an advantageous embodiment it may be the case that the mixture is incubated. The genetically modified microorganisms may accordingly be incubated such that at least one adhesive protein and/or the substances described herein are advantageously overexpressed.

In particular, it may be the case that the mixture is exposed to mechanical pressure during part of or throughout the incubation time. It is preferably the case that the mixture is exposed to an external mechanical pressure, in particular a compression pressure. Mechanical pressure permits particularly high compressive strength in the article being produced.

In an advantageous embodiment it may be the case that the plant-based material is provided in the form of a dry mass. Plant-based material can in particular be comminuted wood and/or wood waste, for example wood shavings and/or cortex materials, especially barks, and/or plant-based fibers, for example hemp, sugar cane, flax or bamboo, and/or straw and/or pomace, preferably wine pomace, and/or wash margin material and/or residual and plant-based by-products of tropical agriculture, for example banana tree waste. The use of a dry mass makes it possible to dispense with chemical treatment of the plant-based material, in particular wood.

Alternatively or in addition, it may be the case that the mixture forms a paste-like mass prior to hardening. A paste-like mass may be formed by adding a liquid, preferably water or the culture medium described herein, to the plant-based material and the genetically modified during microorganisms mixing. It is preferable here that only as much liquid is added to the mixture as is needed for the mixture to become plastically moldable. This is particularly advantageous when shaping of the mixture as described herein is to be carried out.

In an advantageous embodiment it may be the case that at least one stimulus is administered the to genetically modified microorganisms, which modulates the overexpression of the at least one adhesive protein. A stimulus may particularly preferably be an electromagnetic radiation as described herein, in particular the thermal radiation mentioned previously. A stimulus allows the overexpression of the adhesive protein(s) to be selectively modulated, thereby advantageously enabling local and/or temporary overexpression of the adhesive proteins, for example. This allows an article to be produced as required.

In an advantageous embodiment it may be the case that the genetic modification responds to a stimulus that results in overexpression of the adhesive protein. In particular, the process can then also include the initiation of the stimulus as a further process step. The stimulus can be initiated for example before and/or during mixing or else at another time.

It may in particular be the case that more than one stimulus is used in the process, it being possible that each stimulus is administered for modulation of a specific adhesive protein, preferably those described herein. The use of various stimuli thus makes it possible for different adhesive proteins to be overexpressed.

In an advantageous embodiment it may be the case that the mixture undergoes shaping in a mold. This mold can be customized to the shape of the article to be produced, for example through a 3D-printing process. Shaping thus advantageously makes it possible for an article to be shaped in line with user requirements, making the process suitable for the production of a multitude of differently shaped articles.

In particular, it may be the case here that an incubation of the genetically modified microorganisms takes place during shaping. This allows the genetically modified microorganisms to overexpress the at least one adhesive protein during shaping. As a consequence, the components of the mixture can be adhesively bonded particularly efficiently.

The introduction of the mixture into the mold may be designed such that the mixture is introduced into the mold by an injection-molding process or by extrusion. This makes it possible for the mixture to be introduced continuously and evenly.

The introduction of the mixture can also take place in a stepwise manner. This advantageously makes it possible, as may be the case, for the material, in particular the plant-based material described herein, to be added at least partially in the form of a preproduced structural material. Structural material is understood as meaning preferably plant-based material in paper or sheet form which can advantageously improve the use properties of the article. It is accordingly possible, for example through the use of at least one structural material, to improve the strength and/or flexibility of the article.

It may accordingly advantageously be the case that the mixture and at least one structural material are introduced into the mold and that the mixture undergoes shaping in the mold. For instance, a portion of the mixture can advantageously be continuously and evenly introduced into the mold, after which the structural material is positioned on the mixture present in the mold and a second portion of the mixture is transferred onto the structural material. Layering can be carried out with any desired number of layers of structural materials and mixture, thereby allowing a process to be customized with particular versatility to the article to be produced.

In an advantageous embodiment it may be the case that the incubation of the genetically modified microorganisms during shaping is for a period of preferably less than 48 hours. This makes it possible for shaping and adhesive bonding of the components of the mixture to be carried out quickly, making it possible for an article to be produced in a relatively short time.

An article can be produced in an even shorter time when, during the process, shaping is more preferably for a period of less than 24 hours, most preferably for a period of less than 4 hours. These short incubation times during shaping allow a higher throughput during the production of articles.

The duration of incubation during shaping can in particular then be reduced to less than 48, 24 or 4 hours when, as described above, the genetically modified microorganisms are precultured prior to mixing and/or when, as described and claimed hereinbelow, the genetically modified microorganisms are to form at least one substance through the initiation of the first and/or of a or of the second stimulus. This allows a process to be executed quickly and efficiently with a high degree of functional variability.

In an advantageous embodiment it may be the case that the genetically modified microorganisms are cultured prior to shaping at a first incubation temperature and during shaping at a second incubation temperature. This allows the overexpression of an adhesive protein to be improved, especially prior to shaping, very particularly preferably during the preculturing of the genetically modified microorganisms described herein.

In particular, it may be the case here that the first and the second incubation temperature differ by at least 3° C., preferably by at least 12° C. This makes it possible for an optimal temperature difference between the first and second incubation temperature to be realized, making it possible to improve the overexpression of the adhesive protein still further.

In addition, it may in particular be the case that the first incubation temperature is lower than the second incubation temperature. This is because the invention has recognized that the genetically modified microorganisms produce particularly large amounts of adhesive protein especially during the preculturing described herein. This means that the adhesive protein may already be being produced in sufficient amounts prior to mixing, making it possible for a process to be executed particularly quickly.

It may likewise advantageously be the case that the culturing during shaping is spatially limited. This may be realized in particular by the second incubation temperature being applied only in specific regions of the article to be produced. This advantageously makes it possible for the overexpression of an adhesive protein and/or of a substance as described herein and/or the growth and metabolism of the microorganisms to be spatially limited. This allows an article to be designed in a diversity of ways.

In an advantageous embodiment it may be the case that at least one second stimulus is administered to the genetically modified microorganisms. This makes it possible for the genetically modified microorganisms to produce more molecules of an adhesive protein and/or a plurality of different adhesive proteins and/or adhesive proteins and the substance described herein, preferably simultaneously and in particular during the shaping mentioned previously.

In particular, it may be the case here that the first and second stimulus are different in nature. For example, a first stimulus may be the electromagnetic radiation described herein and a second stimulus the sugar derivative mentioned previously. This makes it possible for the overexpression of adhesive proteins and/or substance to be stimulated in different ways, which may be advantageous for example when the adhesive protein and a substance as described herein, for example a coloring agent, are to be overexpressed at different times.

In particular, it may in a process be the case that the two stimuli are administered at different times and/or in different places. This makes it possible for adhesive proteins and/or the substance described herein to be overexpressed at different times and/or in different places, thereby allowing an article to be designed in a particularly wide variety of ways.

In addition, the at least one stimulus may be administered in and/or on a or the mold described herein. This makes it possible to ensure that the stimulus reaches all the genetically modified microorganisms present, including in particular those in the deeper layers of the mixture (i.e. far away from a mold wall).

Preference is given to this execution when the stimulus is the electromagnetic radiation described herein.

In an advantageous embodiment it may be the case that the genetically modified microorganisms form at least one substance through the initiation of the first and/or of a or of the second stimulus. The at least one substance allows the article to have preferred properties. A non-exhaustive description of substrates and the associated properties and advantages is as follows:

A substance may be a substrate crosslinker. A substrate crosslinker is understood as meaning a substance that in particular brings about crosslinking between components of the plant-based material and/or between components and/or further additives of the mixture. This makes it possible for an article to be produced with particular stability.

Particularly preferably, it may be the case that the substrate crosslinker is an enzyme and/or a domain of an enzyme and/or an epitope tag for crosslinking proteins and/or carbohydrates and/or fats and/or other biomolecules in particular.

For example, a cellulose-binding domain can advantageously be used for crosslinking proteins and/or carbohydrates.

For example, the enzyme transglutaminase can advantageously be used for crosslinking proteins.

Alternatively or in addition, a coiled-coil domain known to those skilled in the art can be used as a domain.

Alternatively or in addition, a tyrosinase known to those skilled in the art with optional addition of phenolic compounds can be used as a domain.

Alternatively or in addition, a SpyTag/SpyCatcher or SnoopTag/SnoopCatcher known to those skilled in the art can be used as an epitope tag.

A substance may also be a flame retardant. This makes it possible to prevent/retard combustion of the article. The use of a flame retardant can also make it possible for the article to meet fire-protection requirements, for example in construction and transport and in the electrical and electronics sector, and to be used therein.

For example, proteins such as SR proteins, caseins and/or hydrophobins may advantageously be used as flame retardants. These proteins can be easily produced and provided by genetically modified microorganisms.

For example, organic compounds, in particular polyphenol compounds, may be envisaged as flame retardants. A possible advantage of this is that the polyphenol compounds may for example be extracted cost-effectively from agricultural residues and pomace and added to the mixture described herein.

A substance may also be a biomineralizer. For example, in the process it is possible that a coral-derived protein, preferably silicatein, is overexpressed by the genetically modified microorganisms as a result of the genetic modification. Biomineralizers can be advantageously used as flame retardants, in particular those described and/or claimed herein.

Alternative biomineralization processes using genetically modified microorganisms are also conceivable in the process; biomineralization may advantageously be carried out by Sporosarcinia pasteurii (with sand, urea and calcium) or with Escherichia coli (hydroxyapatide mineralization) or with cyanobacteria (with calcium).

Besides the mentioned genetically modified microorganisms, it is in principle possible in the process to use all genetically modified microorganisms for biomineralization processes. In particular, biomineralization may be carried out by Bacillus sphaericus.

During the biomineralization process, the genetically modified microorganisms absorb supplied nutrients, which are preferably present dissolved in the culture medium described herein. Urea, CO₂, potassium ions, silicate ions, nitrogen compounds, and phosphorus compounds may in particular be present as nutrients, which are transformed into the described biomineralizer(s) during biomineralization.

As a consequence of absorption of the raw materials, the biomineralizers formed may be held by the genetically modified microorganisms in pores and/or cavities in the mixture described herein. To this end, it may be the case that the genetically modified microorganisms are incubated, particularly in an incubation as described herein, in order to be able to optimally carry out the biomineralization. This makes it possible in particular to improve the fire resistance of the article produced/to be produced.

A substance may also be an impregnating agent. This makes it possible for the article to be water-repellant and longer lasting. In particular, the hydrophobins mentioned previously may be overexpressed in the process, thereby resulting in the simultaneous overexpression of an impregnating agent and a flame retardant.

A substance may also be a coloring agent. This coloring agent may in particular be an enzyme that converts a reactant into a reaction product recognizable by its color. Preferred enzymes formed in the process by genetically modified microorganisms may be β-galactosidases, glucuronidases, tyrosinases, and enzymes for the production of pigments, for example violaceins.

A substance may also be a pesticide, as a result of which insects (insecticides), bacteria (bactericides) and/or fungi (fungicides) in particular may be deterred from damaging the finished article. This makes it possible for an article to be designed to be particularly long-lasting, even when used in outdoor areas.

A substance may also be a hydrolytic enzyme, as a result of which the mixture and/or a spatially limited region of the article to be produced can be designed to be soft. In this way an article can advantageously be designed to be softer (or harder) in a spatially limited region than in its other regions.

For example, spores of bacteria, for example spores of Bacillus thuringiensis, may be overexpressed and used for the control of termites (insecticide).

A substance may also be a pore-former. In particular, the genetically modified microorganisms may express in particular enzymes that result in the evolution and release of gases through fermentative processes in particular. Gases can result in the formation of pores in the mixture, as a result of which the density of the article to be produced may be advantageously altered. This makes it possible for the density of an article or of parts of an article to be advantageously adjusted in a process.

Preference can be given to overexpressing/using yeasts, in particular Saccharomyces cerevisiae, and/or yeast proteins as pore-formers. Alternatively or in addition, it is also possible for bacteria to be used and/or for bacterial proteins to be overexpressed. Preferably, enzymes that catalyze a decarboxylation (pyruvate decarboxylase, oxalate decarboxylase) and/or a redox reaction (alcohol dehydrogenase, formate hydrogenase) can be expressed.

It can be said that it is possible to use in a process any substance, in particular any protein, capable of being overexpressed by genetically modified microorganisms and capable of achieving a beneficial effect in the article. For instance, further proteins serving as an active substance can be overexpressed as fragrance, for the breakdown of harmful substances, or as indicator substance for harmful substances, pH or temperature.

In an advantageous embodiment it may be the case that a polysaccharide is added to the mixture. The use of polysaccharides, preferably starch, can improve the connection and adhesive bonding of components of the mixture.

Alternatively or in addition, it may be the case that a protein is added to the mixture. This protein may be one of the proteins mentioned previously, as a result of which the development of particular properties and the associated advantages for the article, in particular those described above, can be improved still further. In particular, the protein may be an enzyme, for example transglutaminase. Alternatively or in addition, a protein mixture, preferably gluten or soy protein, may also be added to the mixture. Gluten and soy protein in particular can adhesively bond synergistically with the adhesive protein components of the mixture described herein, making it possible for an article having particularly high compressive strength to be produced.

Alternatively or in addition, it may be the case that a blowing agent, in particular baking powder, is added to the mixture. The use of a blowing agent can be employed to alter the density of an article to be produced. In particular, a blowing agent, acting synergistically with the pore-formers described herein, can have a particularly beneficial influence on the density of an article. This makes it possible for an article, or sections thereof, to be designed to be particularly soft. This can be particularly advantageous in the case of seating and/or reclining furniture and acoustic panels.

Alternatively or in addition, it may be the case that nanoparticles are added to the mixture. Nanoparticles may be biotic or abiotic in origin. The use of nanoparticles allows the use properties of the article to be improved.

For example, silver nanoparticles may be added to the mixture. An advantage of silver nanoparticles can be their antimicrobial activity, as a result of which undesirable growth of microbial contaminants during the process for producing an article can be prevented. Preference can generally be given also to further antimicrobial nanoparticles for protecting the process from microbial contaminants.

The use of nanoparticles is particularly advantageous when they are produced by the genetically modified and/or wild-type microorganisms, as may be the case.

In an advantageous embodiment it may be the case that a stimulus is an electromagnetic radiation, and wherein initiation of the electromagnetic radiation leads to expression of a or the substance described herein. This makes it possible for an electromagnetic radiation, for example visible light or infrared radiation, to be used to initiate the overexpression of an or the substance described herein. It is also advantageous when the electromagnetic radiation by a radiation source can be initiated locally, for example in a specific region of the mold described herein. This permits local overexpression of a substance (or of an adhesive protein) to be carried out during the process.

In particular, it may be the case that the initiation leads to expression by the genetically modified microorganisms of a or the substrate crosslinker and/or of the flame retardant and/or of the biomineralizer and/or of the impregnating agent and/or of the coloring agent and/or of the pesticide and/or of the pore-former and/or of the hydrolytic enzyme. This makes it possible for an article to exhibit at least the advantages already mentioned that are associated with the expression of the substrate crosslinker and/or of the flame retardant and/or of the biomineralizer and/or of the impregnating agent and/or of the coloring agent and/or of the pesticide and/or of the pore-former and/or of the hydrolytic enzyme. For example, an article may become locally colored when the expressed substance is the or a coloring agent. This advantageously makes it possible for an article to be furnished with lettering and/or a logo, for example, which can boost brand recognition of an article.

Alternatively or in addition, it may be the case that an additive is added to the mixture and is converted into a dye by the coloring agent. This additive may be a reactant as described above. Said reactant can then be converted into a dye by a coloring agent produced by the genetically modified microorganisms and as described herein. Examples of reactants chromogenic glycosides, for example X-Gal (5-bromo-4-chloro-3-indoxyl-β-D-galactopyranoside) or isolated amino acids for the synthesis of pigments, for example the amino acid tyrosine for the synthesis of melanin.

In particular, an additive may act synergistically with individual process subprocesses. For example, coloration of the article using the overexpressed enzyme tyrosinase can be improved by providing further tyrosines as additive in addition to the tyrosines present in the plant-based material. The use of an additive can be advantageous particularly when local coloration, but not coloration of the entire article, is to be achieved.

In an advantageous embodiment it may be the case that microorganisms of another type are provided in addition to the genetically modified microorganisms. For example, wild types may additionally be added to the mixture.

Preference is given to adding genetically modified microorganisms. These additional genetically modified microorganisms may be a different species to the mandatory genetically modified microorganisms added to the process or they may belong to the same species, the difference in nature, particularly in the latter case, being a consequence of the genetic modification. The use of two genetically modified microorganisms of different type can be advantageously utilized as a means of producing two or more different adhesive proteins. The production of one adhesive protein and one substance and/or the production of at least two different substances is also conceivable. It is thus possible for a plurality of components to be produced by genetically modified microorganisms, which is particularly advantageous for the design of an article.

In an advantageous embodiment it may be the case that the hardening of the mixture is executed by an input of heat. Hardening of this nature can be carried out for example in a convection oven or microwave oven. This allows hardening to be carried out in a regulated and particularly swift manner.

In particular, it may be the case that the mixture is baked at a temperature of between 80° C. and 200° C. The invention has recognized that hardening in this temperature range is particularly beneficial for the quality of the article.

In an advantageous embodiment it may be the case that the genetically modified microorganisms are provided in the form of a cell lysate. A cell lysate is understood here as meaning in particular a solution or a suspension containing lysed, i.e. in particular physically and/or chemically and/or osmotically disrupted, cells of the genetically modified microorganisms. A cell lysate comprises preferably mainly, in particular almost exclusively, lysed cells. Genetically modified microorganisms provided in the form of a cell lysate are advantageous particularly when the adhesive protein will not or cannot be secreted through the cell wall of the genetically modified microorganism. A further advantage may be that other molecules present in the cell lysate are able to stabilize the adhesive protein, making it possible for particularly large amounts of adhesive protein to contact the plant-based material during mixing. This makes it possible for the process to be carried out particularly efficiently.

Advantages over isolation of the or of an adhesive protein by methods of isolation and/or purification known to those skilled in the art are, firstly, an increased yield of adhesive protein (due for example to the stabilization of the adhesive protein already brought about in the cell lysate) and, secondly, the shorter process times and associated lower costs.

Alternatively, cell lysate and/or other preferably genetically modified microorganisms may be provided in addition to the genetically modified microorganisms described and/or claimed herein. This makes it possible to provide in particular a combination of cell lysate and a preferably genetically modified microorganism that is maintained in the culture medium and is thus metabolically active. Such a combination brings with it the advantage that other adhesive proteins produced by the preferably genetically modified microorganism and/or the substances described herein are provided in addition to the adhesive protein. This makes it possible for the process to utilize combinations of adhesive protein(s) and substances in a multitude of ways for the production of the article.

Additionally providing (wild-type) microorganisms can be advantageous when the culturing and/or preculturing described herein needs to be cost-effective in design, since special and potentially costly supplements such as antibiotics are not needed for culturing and/or preculturing.

Additionally providing genetically modified microorganisms can advantageously enable the manifold combinations of adhesive protein(s) and substances described above in particular.

Alternatively or in addition, purified proteins may be provided in addition to the genetically modified microorganisms. The proteins are purified here by known methods of purification. Proteins may in particular be the proteins described herein, i.e. very particularly preferably the adhesive protein(s) and/or enzymes. This allows a process for producing an article to be customized with particular versatility and to the pre (culture) conditions. For example, a system as described herein may be provided with specific proteins, such as those mentioned previously, when the microorganisms for the overexpression of said proteins cannot be cultured or can be cultured only poorly in the system.

In an advantageous embodiment it may be the case that at least the stimulus described herein or other stimulus for control of the adhesive protein described herein or other adhesive protein and/or of the substance described herein or other substance is/are initiated. This makes it possible to control in particular the biological function of adhesive proteins and/or substances already assembled in and/or on the cell bodies, as described herein. This advantageously makes it possible for properties of the mixture described herein and/or of the article described herein to be selectively controlled and altered during the process for producing an article. For example, the activity of enzymes described herein can be controlled by a stimulus, for example by the electromagnetic radiation mentioned previously and claimed hereinbelow. This makes it possible, in particular through application of electromagnetic radiation of a particular wavelength, to alter the conformation of the enzyme tyrosinase, thereby making it possible to then employ the tyrosinase for the production of pigments, for example violaceins, in particular as described above.

In particular, the stimulus can be initiated to activate and/or inhibit the adhesive proteins and/or substances.

Activation allows a biological function of a protein, for example the biological function of the adhesive protein described herein and/or the enzyme described herein that has already been assembled in or on the cell bodies of the genetically modified microorganisms, to be activated. This is advantageous particularly when a protein activity is not desired until a particular point in time during the process.

Inhibition allows protein levels, for example levels of adhesive proteins and/or of the enzymes described herein, to be regulated in order, for example, to prevent undesired overproduction of proteins. It is likewise possible for biological functions of proteins to be regulated in analogous manner to the activation described above.

Alternatively or in addition, in order for the mentioned object to be achieved, the features of the coordinate claim directed toward an article are provided according to the invention. In particular, in order to achieve the mentioned object in an article of the type described in the introduction, it is accordingly proposed according to the invention that the article is produced by a process as described above or hereinbelow and/or as claimed hereinbelow. This makes it possible for an article consisting of plant-based material to be produced that, by virtue of the use of microbial adhesive protein, can be free of harmful substances such as formaldehyde. The article according to the invention is accordingly recyclable and not harmful to health, which is particularly advantageous.

Furthermore, the article can be produced cost-effectively, since it can be produced from plant-based materials, such as wood shavings, and through the microbially produced adhesive proteins without large material costs. The article can also be cost-effective when it is monolithic. The monolithic article shaped and hardened in a mold, as described herein, accordingly does not require connecting means such as screws and hinges, for example. A monolithic article can be visually attractive too.

The article may also have particularly high compressive strength, especially when the article is produced through the overexpression of a plurality of adhesive proteins and/or of the substances described herein and preferably with the use of a protein mixture, in particular gluten.

Furthermore, the article according to the invention can be designed to be particularly long-lasting, especially when a pesticide and/or an impregnating agent and/or a flame retardant is overexpressed in the process.

Alternatively or in addition, in order for the mentioned object to be achieved, the features of the coordinate claim directed toward a system for producing the article described and claimed herein are provided according to the invention. In particular, in order to achieve the mentioned object in a system of the type described in the introduction, it is proposed according to the invention that the system comprises an incubator and/or bioreactor and a mold for shaping the article. An incubator and/or a bioreactor encompass all items of apparatus for the culture and incubation of microorganisms known to those skilled in the art. The system according to the invention allows the article described herein to be produced particularly quickly and cost-effectively, especially through the process described herein.

The use of the incubator and/or bioreactor allows the genetically modified microorganisms to be precultured, in particular as described above. This makes it possible for an or the adhesive protein described above and/or a substance as described herein to be overexpressed before the genetically modified microorganisms are mixed with the plant-based material. This allows adhesive bonding of the plant-based components to be carried out particularly quickly.

The use of a mold allows an article to be produced in any desired shape. An advantage of the mold can also be that, as may in particular be the case, the system comprises a preferably interchangeable device for initiating at least one stimulus, the interchangeable device being mounted on or in the mold. This makes it possible for the interchangeable device, which may for example be the abovementioned radiation source, to be guided close to the mixture, with the result that the stimulus is able to penetrate also into the deeper layers of the mixture present in the mold. As a result, the stimulus is able to initiate overexpression of the or of an adhesive protein and/or of a substance described herein locally or throughout. This makes it possible for the production of an article to be particularly improved.

Alternatively or in addition, it may be the case that the system includes a pressure device for exerting the pressure described herein or a mechanical pressure. For example, the pressure device may be a press that exerts preferably external mechanical pressure on the mold and on the mixture, especially while the mixture is being incubated. This makes is possible to design an article being produced to be particularly pressure-resistant.

The system can in addition also include a convection oven and/or microwave oven for hardening, so that hardening and finishing of the article can be carried out quickly.

Alternatively or in addition, in order for the mentioned object to be achieved, the features of the coordinate claim directed toward a use of genetically modified microorganisms are provided according to the invention. In particular, in order to achieve the mentioned object during use of the nature described in the introduction, it is accordingly proposed according to the invention that the adhesive protein effects adhesive bonding of components of the or of a mixture without additional isolation steps. This makes it possible for adhesive bonding of the components of the or of a mixture to be carried out without additional work, for example in the form of isolation steps. An article can accordingly be produced particularly quickly and cost-effectively, especially when the genetically modified microorganisms are used, as may particularly be the case, in a process described herein and/or in a system described herein for the production of an article described herein.

Alternatively or in addition, it may be the case that a stimulus as described herein is employed for the microbial production of the adhesive protein. A stimulus can accordingly be selectively used for overexpression of an adhesive protein that can be utilized directly, and without laborious isolation steps, for adhesively bonding the components of the mixture to produce the or an article.

The invention will now be more particularly described with reference to exemplary embodiments, but is not limited to said exemplary embodiments. Further exemplary embodiments result from combining the features of one or more claims with one another and/or with one or more features of the exemplary embodiments.

In the figures:

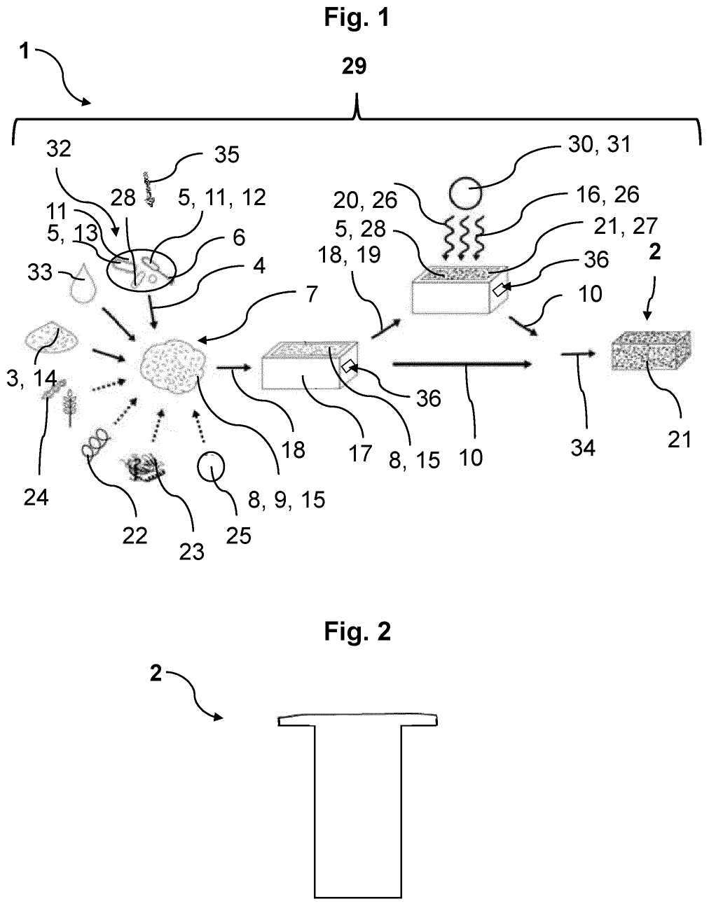

FIG. 1 shows an exemplary embodiment of a system according to the invention in which an article is produced from a plant-based material in an exemplary embodiment of a process according to the invention,

FIG. 2 shows an exemplary embodiment of an article formed according to the invention.

FIG. 1 illustrates a process 1 according to the invention in which an article 2 according to the invention is being produced.

The process 1 is carried out in a system 29 according to the invention, system the 29 including at least an incubator/bioreactor 32 and a mold 17. Also depicted in FIG. 1 is an interchangeable device 30, configured as a radiation source 31, for initiating a first and a second stimulus 16, 20 on the mold 17.

The system 29 further includes a pressure device 36 configured as a press, through which mechanical pressure is externally exerted on the mixture 8 in the mold 17 during incubation of the mixture 8. The pressure device 36 is for better clarity indicated only on one side of the mold 17; the pressure device 36 is however able to mechanically pressurize the mold 17 and thereby the mixture 8 from more than one side in order to exert mechanical pressure on the mixture 8. The mechanical pressure exerted results in the article 2 having particularly high compressive strength.

In an embodiment that is not shown, the interchangeable device 30 is mounted in the mold 17.

As mentioned previously, the radiation source 30, 31 initiates the first and the second stimulus 16, 20, the two stimuli 16, 20 being different electromagnetic radiation 26. In the embodiment shown, the first stimulus 16, 26 is visible light and the second stimulus 20, 20 is infrared radiation. For better illustration, both stimuli 16, 20 are shown in FIG. 1. Depending on use, the first and the second stimulus 16, 20 may be initiated in different places and/or at different times, so that the overexpression of the adhesive protein 6 and/or of a substance 21 is initiated in the desired place(s) and at the desired time(s).

The two stimuli 16, 20 may, as alternatively or additionally desired in the process described hereinbelow, be initiated for the control, in particular for the activation and/or inhibition, of the adhesive protein 6 and/or the substance 21. This allows the regulation in particular of biological functions of the flame retardant described and/or claimed herein and/or of the biomineralizer and/or of the impregnating agent and/or of the coloring agent 27 and/or of the pesticide and/or of the pore-former and/or of the hydrolytic enzyme.

The process 1 illustrated in FIG. 1 proceeds as follows:

The genetically modified microorganisms 5, which have a genetic modification 12 for overexpression of at least one adhesive protein 6, are precultured in a culture medium 11 in the incubator/bioreactor 32. The genetic modification 12 includes a gene sequence 13 that codes for the adhesive protein 6. During this preculturing 35, adhesive proteins 6 will, as a result of the genetic modification 12, already be being overexpressed and assembled on the surface of the genetically modified microorganisms 5. The adhesive protein 6 shown here is a surface structure known as curli fibers. The preculturing 35 and accompanying overexpression of the adhesive proteins 6 allows the process 1 to be carried out particularly quickly.

Alternatively or in addition, it is also possible to provide cell lysate from genetically modified microorganisms 5, in order to supply the adhesive protein 6 and/or at least one substance 21.

In parallel to the preculturing 35 of the genetically modified microorganisms 5, microorganisms 28 of different type are precultured in the or an incubator/bioreactor 32. These microorganisms 28 are likewise genetically modified and in the course of the process overexpress different substances 21 in response to the stimuli 16, 20 already mentioned previously.

After preculturing 35, the genetically modified microorganisms 5, 28 are provided. These may be provided 4 in the culture medium 11, as a result of which the microorganisms 5, 28 can be kept viable and the adhesive proteins 6 kept expressed and intact. The genetically modified microorganisms 5, 28 can here each be provided in a specific culture medium 11 that is tailored to their needs.

After being provided 4, the genetically modified microorganisms 5, 28 are mixed/blended with the plant-based material 3. The plant-based material 3 is here advantageously provided in the form of a dry mass 14 and comprises mainly wood shavings. Blending allows the genetically modified 5, microorganisms 28 to become homogeneously dispersed in the mixture 8, so that the adhesive proteins 6 in particular are optimally distributed for the adhesive bonding 9 of the components of the mixture 8 and distributed in the course of the process 1. The homogeneously dispersed microorganisms 5, 28 likewise allow substances 21 to be evenly overexpressed and dispersed.

During mixing 7, a polysaccharide 22, preferably starch, and a protein 23, preferably at least one enzyme, and a protein mixture 24, in particular gluten or soy protein, and a blowing agent 25, in particular baking powder, are additionally added to the mixture 8 (illustrated in FIG. 1 by dotted arrows pointing toward the mixture 8). The article 2 produced by this process 1 is as a result characterized in particular by high compressive strength. However, a process 1 that dispenses with the additives just mentioned (illustrated in FIG. 1 by solid arrows pointing toward the mixture 8) likewise results in the article 2 having considerable compressive strength.

In addition, liquid 33 is also added to the mixture 8 so that the mixture 8 forms a paste-like mass 15 that is added to the mold 17 for shaping 18. The paste-like mass 8, 15 is incubated, in particular during shaping 18, so that adhesive bonding 9 can continue in an optimal manner during said incubation 19. The incubation 19 illustrated in FIG. 1 is for a period of less than 48 hours, but can alternatively be for a period of less than 24, in particular less than 4, hours. The incubation temperature here, which is a second incubation temperature, is higher for the genetically modified microorganisms 5, 28 than an incubation temperature, which is a first incubation temperature, before shaping 18. In particular, the first incubation temperature during preculturing is at least 3° C. lower (or higher) than the second incubation temperature.

In an embodiment that is not shown, the culturing during shaping 18 may also be spatially limited. For example, it is possible that only one half of the mixture 8 present in the mold 17 is incubated.

In the process shown, the molded mixture 8, 15 is then baked at a temperature of between 80° C. and 200° C. The hardening 10 takes place in a convection oven or alternatively in a microwave oven. This makes it possible for hardening 10 to be carried out relatively quickly.

The system 29 also makes it possible that, following shaping 18, the first and second stimulus 16, 20 are initiated by the previously described radiation source 30, 31, resulting in overexpression by the genetically modified microorganisms 5, 28 of adhesive proteins 6 and substances 21.

By way of example and for better clarity, FIG. 1 shows the substance 21 as being a coloring agent 27 for visually attractive coloring of the article 2. The substance(s) 21 can however also be a substrate crosslinker described herein and/or a flame retardant described herein and/or biomineralizer described herein and/or an impregnating agent described herein and/or a pesticide described herein and/or pore-former described herein and/or another active substance described herein. Through a combination of substances 21 in particular, it is possible to form a particularly vivid and long-lasting article 2. A combination of substances 21 can be produced by the genetically modified microorganisms 5, 28 alone or in combination.

The substance 21 can also be a nanoparticle described herein.

A monolithic article 2 is illustrated in FIG. 2. After hardening 10 and removal 34 from the mold 17, this article 2 was processed further in such a way as to realize the embodiment illustrated in FIG. 2.

In the embodiment according to FIG. 1, the mold 17 is designed such that the two stimuli 16, 20 stimulate the genetically modified microorganisms 5, 28 into overexpressing the substances 21 only locally.

In an embodiment that is not shown, it is also possible that at least one of the stimuli 16, 20 is executed before and/or after shaping 18. In particular, it may be the case that, during preculturing of the genetically modified microorganisms 5, 28, a stimulus 16, 20 is initiated and leads to overexpression of the adhesive protein 6 and/or of the substance 21. This makes it possible to adjust in particular the timing of the overexpression, thereby allowing a process 1 to be carried out in an optimal manner.

In a further embodiment that is not shown, it is possible to ensure, through the choice of stimulus 16, 20 and/or the design of the mold 17 and/or through the application of the interchangeable device 30, that comprehensive overexpression of the substances 21 is carried out.

The invention generally proposes a process 1 for producing an article 2 from a plant-based material 3, comprising the following process steps: providing 4 genetically modified microorganisms 5 in which the genetic modification 12 results in overexpression of at least one adhesive protein 6; mixing 7, preferably blending, of the plant-based material 3 with the genetically modified microorganisms 5, which are preferably held in a culture medium 11, to obtain a mixture 8; expressing of the adhesive protein 6; hardening 10 of the mixture 8.

### LIST OF REFERENCE SIGNS

1 Process
2 Article
3 Plant-based material
4 Providing of 5
Genetically modified microorganisms
6 Adhesive protein
7 Mixing
8 Mixture
9 Adhesive bonding of 8
Hardening of 8
11 Culture medium of 5
12 Genetic modification
13 Gene sequence
14 Dry mass
Paste-like mass
16 Stimulus
17 Mold
18 Shaping
19 Incubation of 5 during 18
Second stimulus
21 Substance
22 Polysaccharide
23 Protein
24 Protein mixture
Blowing agent
26 Electromagnetic radiation
27 Coloring agent
28 Microorganisms of different type to 5
29 System
30 Device
31 Radiation source
32 Incubator/bioreactor
33 Liquid
34 Removal of 2
35 Preculturing
36 Pressure device

1. A process for producing an article from a plant-based material, comprising:
providing genetically modified microorganisms in which the genetic modification results in overexpression of at least one adhesive protein [(6)];
mixing, preferably blending, of the plant-based material with the genetically modified microorganisms, which are preferably held in a culture medium, to obtain a mixture;
expressing of the adhesive protein; and
hardening of the mixture.

2. The process as claimed in claim 1, wherein the genetic modification encompasses a gene sequence that codes for an adhesive protein.

3. The process as claimed in claim 1, wherein the mixture is incubated, in particular wherein the mixture is exposed to mechanical pressure during part of or throughout the incubation time.

4. The process as claimed in claim 1, wherein the plant-based material is provided in the form of a dry mass and/or that the mixture forms a paste-like mass prior to hardening.

5. The process as claimed in claim 1, wherein at least one stimulus is administered to the genetically modified microorganisms, which modulate(s) the overexpression of the adhesive protein(s).

6. The process as claimed in claim 1, wherein the mixture undergoes shaping in a mold, in particular wherein an incubation of the genetically modified microorganisms takes place during shaping.

7. The process as claimed in claim 1, wherein the incubation of the genetically modified microorganisms during shaping is for a period of preferably less than 48 hours, more preferably for a period of less than 24 hours, most preferably for a period of less than 4 hours.

8. The process as claimed in claim 1, wherein the genetically modified microorganisms are cultured prior to shaping at a first incubation temperature and during shaping at a second incubation temperature, in particular wherein the first and the second incubation temperature differ by at least 2° C., preferably by at least 12° C., in particular wherein the first incubation temperature is lower than the second incubation temperature and/or wherein culturing during shaping is spatially limited.

9. The process as claimed in claim 1, wherein at least one second stimulus is administered to the genetically modified microorganisms, in particular wherein the first and second stimulus are different in nature, wherein the two stimuli are in particular administered at different times and/or in different places, in particular wherein at least one stimulus is administered in and/or on a or the mold.

10. The process as claimed in claim 1, wherein the genetically modified microorganisms form at least one substance through the initiation of the first and/or of a or of the second stimulus that serve(s) in particular as substrate crosslinker and/or as flame retardant and/or as biomineralizer and/or as impregnating agent and/or as coloring agent and/or as pesticide and/or as pore-former and/or as hydrolytic enzyme.

11. The process as claimed in claim 1, wherein a polysaccharide, preferably starch, and/or a protein, preferably at least one enzyme, and/or a protein mixture, in particular gluten or soy protein, and/or a blowing agent, in particular baking powder, and/or nanoparticles, particularly preferably silver nanoparticles, is/are added to the mixture.

12. The process as claimed in claim 1, wherein a stimulus is an electromagnetic radiation, and wherein the initiation of the electromagnetic radiation leads to overexpression of a or the substance, in particular wherein the initiation leads to overexpression by the genetically modified microorganisms of a or the substrate crosslinker and/or of the flame retardant and/or of the biomineralizer and/or of the impregnating agent and/or of the coloring agent and/or of the pesticide and/or of the pore-former and/or of the hydrolytic enzyme and/or wherein an additive is added to the mixture and is converted into a dye by the coloring agent.

13. The process as claimed in claim 1, wherein microorganisms of another type, preferably genetically modified, are provided in addition to the genetically modified microorganisms.

14. The process as claimed in claim 1, wherein the hardening of the mixture is executed by an input of heat, in particular wherein the mixture is baked at a temperature of between 80° C. and 200° C.

15. The process as claimed in claim 1, wherein the genetically modified microorganisms are provided in the form of a cell lysate or that cell lysate and/or other preferably genetically modified microorganisms and/or purified proteins are provided in addition to the genetically modified microorganisms.

16. The process as claimed in claim 1, wherein at least the or a stimulus for the control, in particular for the activation and/or inhibition, of the or of an adhesive protein and/or of the or of a substance is/are initiated.

17. An article, which is preferably monolithic, produced by a process as claimed in claim 1.

18. A system for producing an article as claimed in claim 17, comprising:
an incubator and/or bioreactor;
a mold for shaping the article; and
a preferably interchangeable device for initiating at least one stimulus, the interchangeable device being mounted on or in the mold;
and/or wherein the system includes a pressure device for applying the or a mechanical pressure.

19. (canceled)

20. A method of producing an adhesive protein, comprising:
providing genetically modified microorganisms for the microbial production of an adhesive protein; and
effecting adhesive bonding of components of a mixture with the adhesive protein without additional isolation steps.
